(19) United States
(12) Patent Application Publication
(10) Pub. No.: US 2019/0105698 A1
(43) Pub. Date: Apr. 11, 2019

(54) METHOD OF PRODUCING A DENSELY PERFORATED TIPSHELL FROM FLAT GEOMETRY

(71) Applicant: BIOSENSE WEBSTER (ISRAEL) LTD., Yokneam (IL)

(72) Inventors: Christopher Thomas Beeckler, Brea, CA (US); Joseph Thomas Keyes, Glendora, CA (US)

(21) Appl. No.: 15/730,223

(22) Filed: Oct. 11, 2017

(51) Int. Cl. B21D 22/28; B21D 35/00; A61B 18/14

(52) U.S. Cl. CPC B21D 22/28; A61B 2018/00577; A61B 18/1492; B21D 35/001

(57) ABSTRACT

A manufacturing method includes providing a flat sheet perforated with holes, which have respective initial shapes and respective axes of symmetry. The axes of symmetry of the holes point toward a common origin within the flat sheet. The perforated flat sheet is deep-drawn into a cylindrical shell using a punch centered about the common origin, including deforming the holes from the initial shapes into respective predefined final shapes having different aspect ratios relative to the initial shapes.

### FIELD OF THE INVENTION

The present invention relates generally to medical probes, and particularly to design and manufacturing of irrigated ablation catheters.

### BACKGROUND OF THE INVENTION

Irrigated electrode catheters comprising perforated tip-shells are in common use for cooling tissue during ablation. For example, U.S. Pat. No. 9,510,894 describes an irrigated ablation catheter that includes a tip electrode with a thin shell and a plug to provide a plenum chamber. The tip electrode has an inlet of a predetermined size and noncircular shape, and outlets in the form of fluid ports formed in the thin shell wall. The plurality of the fluid ports is predetermined, as is their diameter. Each fluid port has a tapered configuration, for example, a run-down conical configuration, with a smaller inlet diameter and a larger outlet diameter.

U.S. Pat. No. 9,089,932 describes forming one or more holes through a portion of a housing. The shape of the housing component may be at least partially created by deep drawing portions of a sheet of metal. The sheet of metal may include holes comprising any suitable size and shape, including, but not limited to, triangular, rectangular, elliptical, etc.

U.S. Pat. No. 9,434,025 describes a method for drilling a hole through a component. The method includes a step of applying a pulse laser beam to drill a section of the hole substantially within a thermal barrier coating of the component in a direction substantially perpendicular to a top surface of the component. A further step is conducted to apply the pulse laser beam to further drill through a base metal of the component to complete the formation of the hole extending through the component.

U.S. Pat. No. 5,026,965 describes the provision of a tube with precise holes. The side of a tube is drilled at predetermined intervals by a laser beam while the tube is being conveyed in its longitudinal direction in a tubular state. In consequence, manufacture is facilitated, and the precise holes can be drilled.

### SUMMARY OF THE INVENTION

An embodiment of the present invention provides a manufacturing method including providing a flat sheet perforated with holes, which have respective initial shapes and respective axes of symmetry. The axes of symmetry of the holes point toward a common origin within the flat sheet. The perforated flat sheet is deep-drawn into a cylindrical shell using a punch centered about the common origin, including deforming the holes from the initial shapes into respective predefined final shapes having different aspect ratios relative to the initial shapes.

In some embodiments, deforming the holes includes one or more of (i) elongating the holes along the axes of symmetry, and (ii) compressing the holes perpendicularly to the axes of symmetry. In some embodiments, the initial shapes of the holes that are elliptical. In an embodiment, the axes of symmetry of the holes include minor axes of elliptical holes, axes perpendicular to the axes of symmetry include major axes of the elliptical holes, and deforming the holes includes deforming the elliptical holes into respective circular holes.

In an embodiment, holes that are distributed along different concentric circles surrounding the common origin have different respective initial shapes. In some embodiments, the method includes assembling the cylindrical shell in a distal end of a medical instrument, in which the holes serve as irrigation orifices.

There is additionally provided, in accordance with an embodiment of the present invention, an article of manufacture, including a flat sheet perforated with holes, which have respective elliptical shapes.

There is also provided, in accordance with an embodiment of the present invention, a method of design including, based on a predefined deep-drawing process that uses a predefined punch, designing holes in a flat sheet, wherein the holes have respective initial shapes and respective axes of symmetry pointing toward a common origin within the flat sheet. The holes are designed such that deep-drawing the perforated flat sheet into a cylindrical shell using the predefined punch centered about the common origin will deform the holes from the initial shapes into respective predefined final shapes having different aspect ratios relative to the initial shapes.

The present invention will be more fully understood from the following detailed description of the embodiments thereof, taken together with the drawings in which:

### BRIEF DESCRIPTION OF THE DRAWINGS

FIG. 1 is a schematic, pictorial illustration of a system for cardiac ablation therapy, in accordance with an embodiment of the present invention;

FIG. 2 is a schematic, pictorial illustration of a perforated sheet comprising elliptical holes, for producing a catheter tip-shell, in accordance with an embodiment of the present invention; and

FIGS. 3A-3C are schematic pictorial illustrations showing successive stages of a deep-drawing process for manufacturing a catheter tip-shell that comprises circular irrigation holes, in accordance with an embodiment of the present invention.

### DETAILED DESCRIPTION OF EMBODIMENTS

### Overview

Catheters comprising tip-shell electrodes fitted at their distal end are commonly used for ablation. The tip-shell electrode allows ablating along a curve, where the tip-shell electrode is repositioned repeatedly or dragged across the tissue along the curve. Irrigated ablation tip-shell electrodes are in regular use, aimed at reducing tissue temperature during ablation as to minimize the formation of char and coagulum. The shape of irrigation holes and their distribution over the tip-shell electrode play a major role in the efficiency of tissue cooling, which at the same time requires maintaining low cooling fluid load on the patient body. This constraint is especially emphasized during cardiac ablation treatments.

A demanding processing step in the manufacturing of a quality perforated tip-shell electrode is the formation of accurate irrigation holes in a tip-shell cylinder. One possible way of forming accurate irrigation holes in a blank cylindrical shell is to use precise drilling techniques, which may utilize laser cutting. This challenging accurate cutting, which has to be performed through the three-dimensional curved object, requires complicated tooling and procedures for the alignment and the cutting.

Embodiments of the present invention that are described herein provide improved methods for designing and manufacturing perforated tip-shell electrodes. The design and manufacturing typically comprises forming the irrigation holes without the need to fixture and accurately manipulate individual parts to cut the holes during such a later manufacturing stage, after the cylindrical shell was already made. Rather, in some embodiments, a flat sheet blank is cut with certain initial hole geometries and then deep-drawn into the cylindrical shell, such that the required final hole geometries are formed in the process of deep-drawing.

In some embodiments, a blank disc is provided, and elliptical holes are cut through the disc. The elliptical holes are distributed over circumferences sharing a common origin. The minor axes of the elliptical holes are all aligned along radial directions pointing to the common origin, and the major axes of the elliptical holes are tangent to the circumferences. In a subsequent deep-draw manufacturing step, a punch, centered about the origin, is pressed against the blank disk and deforms the disk into the cylindrical shell. The deformation causes the elliptical holes to elongate along their minor axes and/or compress along their major axes, so as to deform the elliptical holes into circular holes in the cylindrical shell, as required.

In some embodiments, the holes in the cylindrical shell may have any desired predefined final shapes provided the respective holes in the flat sheet are having respective axes of symmetry pointing toward a common origin. The predefined final shapes include circles, ellipses, irregular circles, regular or irregular polygons, and ‘amoebic’ shapes, for example, kidney-bean, crescent, peanut, hourglass, and pear shapes, to name to name only a few. To achieve those predefined final shapes, the respective initial shapes of the holes in the flat sheet are designed with respective axes of symmetry pointing toward a common origin, and take into account the subsequent elongation along the radial directions and/or compression along the tangential directions caused by the deep-draw process.

In an optional embodiment, the irrigation holes are formed in ring-shell electrodes, using a manufacturing process similar to that employed in forming irrigation holes in tip-shell electrodes.

The disclosed technique provides a manufacturing process that can substantially reduce the cost of manufacturing of perforated shells. Moreover, the disclosed techniques open possibilities to design and manufacture of complex perforated cylindrical shells.

### System Description

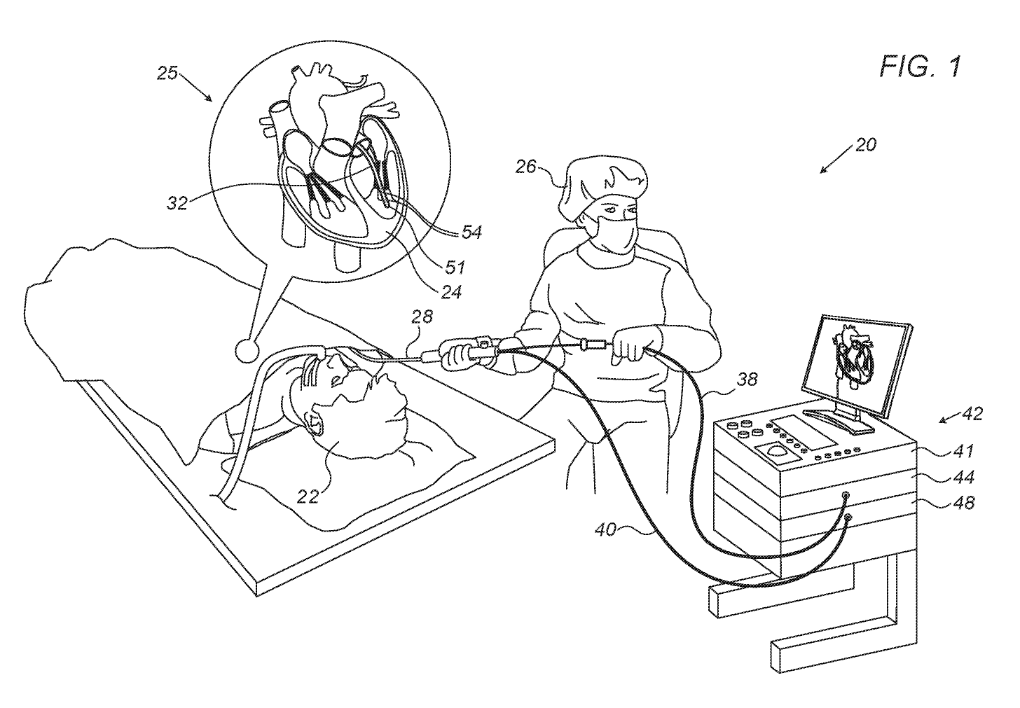

FIG. 1 is a schematic, pictorial illustration of a system 20 for cardiac ablation therapy, in accordance with an embodiment of the present invention. An operator 26 inserts a catheter 28 through a blood vessel into a chamber of a heart 24 of a subject 22, and manipulates the catheter so that a distal end 32 of the catheter contacts the endocardium in an area that is to be treated. A distal tip shell 51 of catheter 28 is perforated with circular holes 54, seen in inset 25, to enable optimal irrigation of the treatment area. Techniques for manufacturing such tip shells are described below.

After positioning distal end 32 at an ablation site, and ensuring that the tip is in contact with the endocardium at the site, operator 26 actuates a Radio Frequency (RF) energy generator 44 in a control console 42 to supply RF energy via a cable 38 to distal end 32. Meanwhile, an irrigation pump 48 supplies a cooling fluid, such as normal saline solution, via a tube 40 and a lumen in catheter 28 to the distal end. Operation of the RF energy generator and the irrigation pump may be coordinated in order to give the appropriate volume of irrigation during ablation, so as to cool the tip of the catheter and the tissue without overloading the heart with irrigation fluid. A temperature sensor (not shown in the figures) in distal end 32 may provide feedback to console 42 for use in controlling the RF energy dosage and/or irrigation volume.

Although the pictured embodiment relates specifically to the use of a tip ablation device for ablation of heart tissue, the methods described herein may alternatively be applied in other sorts of ablation devices, such as single-arm and multi-arm ablation devices comprising tip-shell and/or ring-shell electrodes having irrigation holes.

### Producing a Densely Perforated Tipshell From Flat Geometry

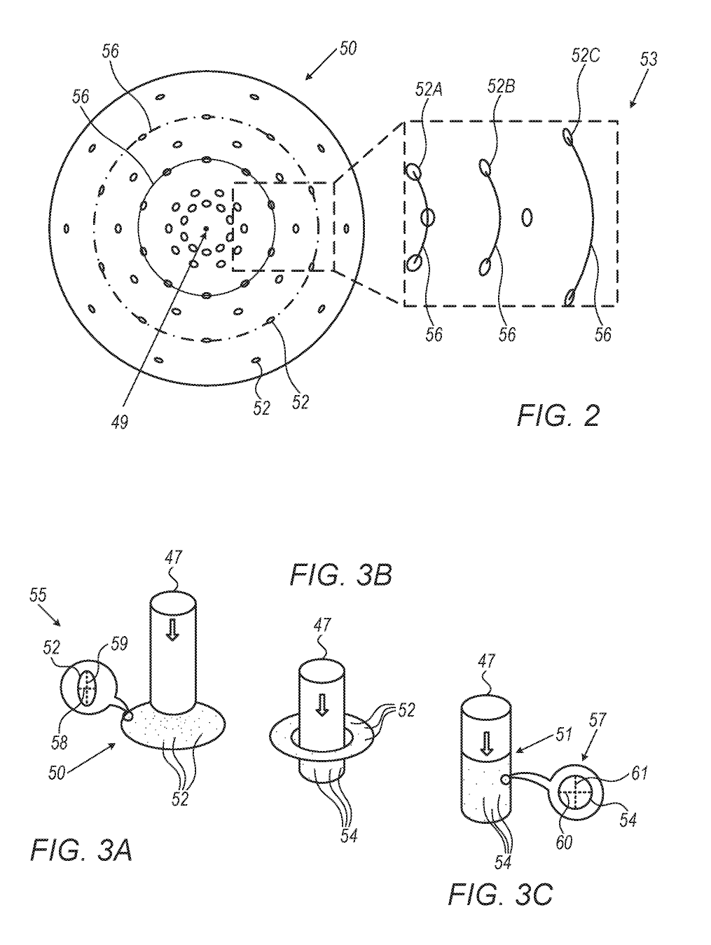

FIG. 2 is a schematic, pictorial illustration of a perforated flat round sheet 50 comprising elliptical holes 52, for producing a catheter tip-shell, in accordance with an embodiment of the present invention. In the figure, flat round sheet 50 is already provided perforated with elliptical holes 52. In an example embodiment, holes 52 are pre-cut in a manufacturing process that typically applies laser cutting through blank flat round sheet 50 (cutting not seen in figures). Alternatively, holes may be formed by a chemical etching process or an Electrical Discharge Machining (EDM) process.

In some embodiments, elliptical holes 52, which have respective initial elliptical shapes, are cut along respective different concentric circles surrounding the common origin 49, such as along outlined circles 56. This is demonstrated in an inset 53 that enlarges part of perforated flat round sheet 50. As seen, elliptical holes 52A, 52B and 52C (which are distributed along different concentric circles 56) have indeed different respective initial elliptical shapes.

In general, holes 52 have their minor axes (Seen in FIG. 3A), which are also the axes of symmetry of elliptical holes 52, pointing toward an origin 49 of perforated flat round sheet 50, i.e., in radial directions. The major axes of elliptical holes 52 (Seen in FIG. 3A) are thus tangent to circles that share common origin 49.

The design of elliptical holes 52 takes into account subsequent deformation that elliptical holes 52 would undergo during a deep-draw manufacturing process. Namely, the eccentricity of the elliptical holes 52 is designed so that elliptical holes 52 of sheet 50 will transform into circular holes in a shell, as described below.

The example configuration shown in FIG. 2 is chosen purely for the sake of conceptual clarity. Other initial shapes and respective axes of symmetry are possible, as long as the axes of symmetry of the holes point toward a common origin within flat sheet 50. Other arrangements of holes 52 over sheet 50 are possible. For example, the density and/or the sizes of holes may vary.

In some embodiments, holes of different initial shapes are distributed along different respective concentric circles surrounding the common origin as to have all the same final shapes. Alternatively, in an optional embodiment, dissimilar final shapes of the holes may be designed, for example, by varying aperture sizes as a function of a radial distance from common origin 49. In an optional embodiment, any hole geometry can be created so long as there is an inverse flat-solution of the geometry (e.g., a square could be created from a rectangle).

FIGS. 3A-3C are schematic pictorial illustrations showing successive stages of a deep-drawing manufacturing process for manufacturing a catheter tip-shell 51 that comprises circular irrigation holes 54, in accordance with an embodiment of the present invention. As seen in FIG. 3A, a punch 47 is pressed against perforated flat round sheet 50, causing sheet 50 to deform into shell 51. FIG. 3B shows the deep-drawing process at an intermediate stage, where part of sheet 50 has already been transformed into shell 51. As a result, holes closer to the origin are transformed into circular holes 54, and holes further away from the origin are still elliptical. As shown in FIG. 3C, the deformation causes elliptical holes 52 to be deformed along their minor axes and/or their major axes, thus elongating along their minor axes and/or compressing along their major axes, respectively, and producing circular holes 54, as described in detail below.

An inset 55 of FIG. 3A shows one elliptical hole 52 (before the deep draw process) in detail. As seen, elliptical hole 52 has a minor axis 58 and a major axis 59. An inset 57 of FIG. 3C shows circular hole 54 (after the deep draw process). As seen, radial axis 60 (previously minor axis 58 of elliptical hole 52) and the perpendicular, tangential axis 61 (previously major axis 59 of elliptical hole 52) are now equal or nearly equal in size.

The change in shape that elliptical holes 52 undergo during a deep-draw process is in general a change in aspect ratio of the ellipses. In the context of the present patent application and in the claims the term “aspect ratio of a hole” is defined as the ratio between the longest length of the hole along the axis of symmetry of the hole that points at the common origin and the longest length along the axis perpendicular to the axis of symmetry. (The cylindrical curving of holes while being deep-drawn is irrelevant to this definition as it occurs along a third, orthogonal axis.) In the specific case of an ellipse, the aspect ratio is the ratio between the lengths of the ellipse's minor and major axes.

The example configuration shown in FIG. 3 is chosen purely for the sake of conceptual clarity. Other respective predefined final shapes of holes 54 in shell 51 are possible, different from the respective initial shapes of holes 52, while still using the disclosed design and manufacturing principles. These predefined final shapes are the deep-drawn result of certain initial-shapes of perforated holes 52 in sheet 50.

Namely, any predefined final shapes are possible to achieve, as long as they are obtained by deforming holes from their initial shapes into respective predefined final shapes while modifying the aspect ratios. Examples of such possible predefined final shapes of holes in shell 51 include ‘amoebic’ shapes such as kidney-bean, crescent, peanut, hourglass, and pear shapes, to name only a few.

The disclosed technique of producing irrigation holes may also be used in other types of electrodes, such as ring-shell electrodes. The processing steps will be similar to those employed in forming irrigation holes in tip-shell electrodes. Additional steps may be employed, for example cutting off the base of the tip-shell to form a ring, and finishing such as wedging, curving and/or smoothing the ring edges.

Although the embodiments described herein mainly address design and manufacturing of cylindrical shells for irrigated tip-shell electrodes for cardiac applications, the methods described herein can also be used in other medical and non-medical applications. For example, the disclosed techniques can be used with the design and manufacturing other suitable distal-end electrode assemblies that comprise perforated cylindrical shells, such as multi-arm and basket catheters.

It will thus be appreciated that the embodiments described above are cited by way of example, and that the present invention is not limited to what has been particularly shown and described hereinabove. Rather, the scope of the present invention includes both combinations and sub-combinations of the various features described hereinabove, as well as variations and modifications thereof which would occur to persons skilled in the art upon reading the foregoing description and which are not disclosed in the prior art. Documents incorporated by reference in the present patent application are to be considered an integral part of the application except that to the extent any terms are defined in these incorporated documents in a manner that conflicts with the definitions made explicitly or implicitly in the present specification, only the definitions in the present specification should be considered.

1. A manufacturing method, comprising:
providing a flat sheet perforated with holes, which have respective initial shapes and respective axes of symmetry, wherein the axes of symmetry of the holes point toward a common origin within the flat sheet; and
deep-drawing the perforated flat sheet into a cylindrical shell using a punch centered about the common origin, including deforming the holes from the initial shapes into respective predefined final shapes having different aspect ratios relative to the initial shapes.

2. The manufacturing method according to claim 1, wherein deforming the holes comprises one or more of (i) elongating the holes along the axes of symmetry, and (ii) compressing the holes perpendicularly to the axes of symmetry.

3. The manufacturing method according to claim 1, wherein the initial shapes of the holes are elliptical.

4. The manufacturing method according to claim 3, wherein the axes of symmetry of the holes comprise minor axes of elliptical holes, wherein axes perpendicular to the axes of symmetry comprise major axes of the elliptical holes, and wherein deforming the holes comprises deforming the elliptical holes into respective circular holes.

5. The manufacturing method according to claim 1, wherein holes that are distributed along different concentric circles surrounding the common origin have different respective initial shapes.

6. The manufacturing method according to claim 1, and comprising assembling the cylindrical shell in a distal end of a medical instrument, in which the holes serve as irrigation orifices.

7. An article of manufacture, comprising a flat sheet perforated with holes, which have respective elliptical shapes whose minor axes point toward a common origin within the flat sheet.

8. The article of manufacture according to claim 7, wherein holes that are distributed along different concentric circles surrounding the common origin have different respective initial elliptical shapes.

9. The article of manufacture according to claim 7, wherein the flat sheet comprises a disc.

10. A method of design, comprising:
based on a predefined deep-drawing process that uses a predefined punch, designing holes in a flat sheet, wherein the holes have respective initial shapes and respective axes of symmetry pointing toward a common origin within the flat sheet, such that deep-drawing the perforated flat sheet into a cylindrical shell using the predefined punch centered about the common origin will deform the holes from the initial shapes into respective predefined final shapes having different aspect ratios relative to the initial shapes.

11. The method according to claim 10, wherein deep-drawing the sheet will deform the holes by one or more of (i) elongating the holes along the axes of symmetry, and (ii) compressing the holes perpendicularly to the axes of symmetry.

12. The method according to claim 10, wherein designing the holes comprises designing the respective initial shapes of the holes to be elliptical.

13. The method according to claim 12, wherein designing the respective initial shapes of the holes to be elliptical comprises designing the respective axes of symmetry of the holes to comprise minor axes of elliptical holes, and axes perpendicular to the axes of symmetry to comprise major axes of the elliptical holes, and wherein deep-drawing the sheet will deform the holes comprises deforming the elliptical holes into respective circular holes.

14. The method according to claim 10, wherein designing the holes comprises distributing holes along different concentric circles surrounding the common origin, the holes having different respective initial shapes.

15. The method according to claim 10, and comprising assembling the cylindrical shell in a distal end of a medical instrument, in which the holes serve as irrigation orifices.
